(19) United States
(12) Patent Application Publication
(10) Pub. No.: US 2019/0231175 A1
(43) Pub. Date: Aug. 1, 2019

(54) MULTI-LUMEN CAP

(71) Applicant: UVision360, Inc., Research Triangle Park, NC (US)

(72) Inventors: Erich Dreyer, Durham, NC (US); Allison London Brown, Durham, NC (US); David Robinson, Schaumburg, IL (US)

(21) Appl. No.: 15/885,108

(22) Filed: Jan. 31, 2018

(51) Int. Cl. A61B 1/00; A61B 1/018; A61B 1/05; A61B 1/07

(52) U.S. Cl. CPC A61B 1/00137; A61B 1/018; A61B 1/07; A61B 1/05; A61B 1/00186

(57) ABSTRACT

Various multi-lumen caps and methods of use are provided herein that can be integrated with devices configured to image target tissue from a body cavity. In an exemplary embodiment, an imaging sleeve is provided with an elongate shaft having distal and proximal ends. The elongate shaft is configured to receive at least a portion of an imaging device therein, and an engagement member is disposed on the proximal end and configured to engage with the imaging device. A cap is configured to removably attach to the distal end of the elongate shaft and engage each of the plurality of lumens. The cap is configured to seal one or more of the plurality of lumens and to leave open one or more of the plurality of lumens.

### FIELD

Devices, methods, and systems of using a multi-lumen cap are provided herein.

### BACKGROUND

Minimally invasive surgeries, diagnostic procedures, exploratory procedures, and other medical procedures have been favored more and more by patients and physicians given the improved healing times and the less invasive nature of the operations. Various medical devices and instrumentation have been developed to accomplish these operations, such as medical introducers, imaging devices such as fiber optic scopes, and other related endoscopic devices.

For minimally invasive surgeries to be successful, however, devices with elongate shafts must be inserted into a patient and maneuvered to a target site within the patient's body. The shafts often have one more lumens therethrough, and the lumens may or may not be needed for any particular operation. However, designing, manufacturing, and delivering devices with specialized elongate shafts for every operation increases the cost and complexity of operations.

Accordingly, there remains a need for improved devices, systems, and methods of use of multi-lumen caps for use therein.

### SUMMARY

Devices, methods, and systems of using a multi-lumen cap are provided herein. In one embodiment, an imaging sleeve is provided that has an elongate shaft with distal and proximal ends, a plurality of lumens extending therebetween, and a longitudinal axis extending therealong. The elongate shaft is configured to receive at least a portion of an imaging device therein. An engagement member is positioned on the proximal end of the elongate shaft and is configured to engage with the imaging device. A cap is configured to removably attach to the distal end of the elongate shaft and is configured to engage each of the plurality of lumens. The cap is configured to seal one or more of the plurality of lumens and to leave open one or more of the plurality of lumens. The cap thus either seals or leaves open each of the plurality of lumens.

The imaging sleeve can vary in numerous ways. For example, one or more protuberances can extend proximally from a proximal-facing surface of the cap and can be configured to correspond with and leave open one or more of the plurality of lumens. One or more openings can extend through the cap and can be configured to correspond with and seal one or more of the plurality of lumens. The imaging sleeve can also include a viewing lumen extending through the elongate shaft and a corresponding viewing channel extending through the cap. The viewing lumen can be configured to receive the imaging device therein. In another example, a distal end of the viewing channel of the cap is sealed and has a window disposed therein that is configured to allow imaging therethrough by the imaging device. In one embodiment, the cap can be configured to be asymmetrical. In another embodiment, the cap can be configured to be symmetrical. The cap can have a blunt, conical shape, and the cap can have no sharp edges thereon. The cap can also attach to the distal end of the elongate shaft through a friction fit. In one example, the cap is formed through injection molding, 3D printing, extruded then tipped, and/or mechanical construction. The imaging sleeve can include fluid in and fluid out lumens that can extend through the elongate shaft and can be configured to deliver and remove fluid through the elongate shaft and to the distal end thereof. The cap can be configured to seal the fluid in and fluid out lumens. The imaging sleeve can also include a working lumen extending through the elongate shaft that is configured to removably receive one or more surgical tools therethrough. The cap can be configured to leave open the working lumen. The engagement member can be configured to prevent relative movement between the imaging sleeve and the imaging device.

In another aspect, an imaging system is provided that includes an imaging sleeve with an elongate shaft that has distal and proximal ends and a plurality of lumens extending therebetween. The imaging sleeve has an engagement member that is positioned on the proximal end of the elongate shaft, and the imaging sleeve has a cap that is removably positioned on the distal end of the elongate shaft. The cap is configured to seal at least one of the plurality of lumens. The system also includes an imaging device with a handle and an elongate imaging member extending distally from the handle. The elongate imaging member is configured to be inserted into one of the plurality of lumens of the elongate shaft of the imaging sleeve, and the elongate imaging member is configured to take images therefrom. The handle is configured to engage with the engagement member of the imaging sleeve.

The imaging system can have a variety of different embodiments. For example, the lumen configured to receive the elongate imaging member through the elongate shaft can be a viewing lumen, and a corresponding viewing channel can extend through the cap. The viewing channel can be configured to allow imaging by the elongate imaging member therethrough. A distal end of the viewing channel of the cap can be sealed and can have a window disposed therein that is configured to allow imaging therethrough by the elongate imaging member. In another example, one or more protuberances can extend proximally from a proximal-facing surface of the cap and can be configured to correspond with and seal one or more of the plurality of lumens. One or more openings can extend through the cap and can be configured to correspond with and leave open one or more of the plurality of lumens. In one embodiment, the cap can have a blunt, conical shape. In another embodiment, the engagement member can be configured to prevent relative movement between the imaging sleeve and the imaging device. The imaging device can be a Complementary Metal-Oxide Semiconductor fiberscope.

In another aspect, a method of imaging a tissue sample is provided that includes attaching a cap onto a distal-most end of an imaging sleeve such that at least one of a plurality of lumens extending through the imaging sleeve is sealed by the cap. The method also includes inserting the cap and the imaging sleeve into a body cavity adjacent to tissue to be imaged. The method further includes imaging a tissue sample using an imaging device disposed at least partially in the imaging sleeve. The imaging device images the tissue through a window on the cap on the imaging sleeve.

The method can have several variations. For example, the method can include sealing a viewing lumen that extends through the imaging sleeve by positioning a viewing channel and window in the cap to positionally correspond with the viewing lumen to allow the imaging device to image through the window, wherein the imaging device is at least partially disposed in the viewing lumen. In another example, the method can further include sealing fluid in and fluid out lumens that extend through the imaging sleeve by positioning protuberances on the cap into the lumens such that irrigation cannot be applied therethrough. The method can also include positioning an opening in the cap to correspond to a working lumen that extends through the imaging sleeve, and passing a surgical tool through the working lumen and the opening to a surgical site.

In another aspect, a cap is provided that has a body with a flat bottom surface and a conical dome. At least one channel extends from the bottom surface, through the body, and out of the conical dome. The flat bottom surface has at least one protuberance extending therefrom. The flat bottom surface is configured to be removably attached to a distal end of an elongate shaft of a surgical tool, and the at least one protuberance is configured to seal at least one lumen extending through the elongate shaft. The at least one channel is configured to align with at least one lumen extending through the elongate shaft.

The cap can be varied in numerous ways. For example, the body can extend to a blunt, distal point. A distal end of the at least one channel can also be sealed and can have a window disposed therein that is configured to allow imaging therethrough. Additionally, the cap can be configured to be asymmetrical or can be configured to be symmetrical. The cap can also have no sharp edges thereon. In another example, the cap can be formed through injection molding, 3D printing, extruded then tipped, and/or mechanical construction.

### BRIEF DESCRIPTION OF THE DRAWINGS

The invention will be more fully understood from the following detailed description taken in conjunction with the accompanying drawings, in which:

FIG. 1 is a side view of one embodiment of an imaging sleeve with an imaging device;

FIG. 2 is a side view of the imaging device of FIG. 1;

FIG. 3 is a side view of the imaging sleeve of FIG. 1;

FIG. 4 is a distal-to-proximal view of a distal tip of the imaging sleeve of FIG. 1;

FIG. 5 is a side perspective view of one embodiment of a multi-lumen cap;

FIG. 6 is a bottom perspective view of the multi-lumen cap of FIG. 5;

FIG. 7 is a perspective view of the multi-lumen cap of FIG. 5 on the distal end of the imaging sleeve of FIG. 1;

FIG. 8 is a side perspective view of another embodiment of a multi-lumen cap;

FIG. 9 is a bottom perspective view of the multi-lumen cap of FIG. 8; and

FIG. 10 is a perspective view of the multi-lumen cap of FIG. 8 on a distal end of a shaft.

### DETAILED DESCRIPTION

Certain exemplary embodiments will now be described to provide an overall understanding of the principles of the structure, function, manufacture, and use of the devices and methods disclosed herein. One or more examples of these embodiments are illustrated in the accompanying drawings. Those skilled in the art will understand that the devices and methods specifically described herein and illustrated in the accompanying drawings are non-limiting exemplary embodiments and that the scope of the present invention is defined solely by the claims. The features illustrated or described in connection with one exemplary embodiment may be combined with the features of other embodiments. Such modifications and variations are intended to be included within the scope of the present invention.

A variety of procedures use an apparatus with an elongate shaft and a plurality of lumens, such as a sleeve and an imaging device used in non-invasive operations. For example, a physician may be required to accurately image a body cavity within a patient, such as when imaging uterine tissue. However, the sleeve may have a less-than-desirable configuration, such as having multiple lumens that are not needed. For instance, a sleeve might be designed as a generic sleeve to be used in multiple operations, thus saving time and money on the sleeve but resulting in unused lumens. Multi-lumen caps are thus provided herein that can be attached to a variety of elongate shafts with a plurality of lumens extending therethrough. The multi-lumen caps are configured to seal one or more of the lumens and leave open or unblocked others of the lumens. This flexibility allows a user to create any configuration of sealed and unsealed lumens of an apparatus with an elongate shaft that contains more than one lumen or channel therethrough. While various medical sleeves are described herein with imaging devices, such as fiberscopes for use on uterine tissue, the multi-lumen caps described herein can be used with a variety of apparatuses with more than one lumen and are not limited to any one apparatus, sleeve, and/or imaging device.

In an exemplary embodiment, a sleeve is provided with an elongate shaft having distal and proximal ends. The elongate shaft is configured to receive at least a portion of an imaging device therein, and an engagement member is disposed on the proximal end and configured to engage with the imaging device. A cap is configured to removably attach to the distal end of the elongate shaft and engage each of the plurality of lumens such that the cap seals one or more of the plurality of lumens and leaves open one or more of the plurality of lumens.

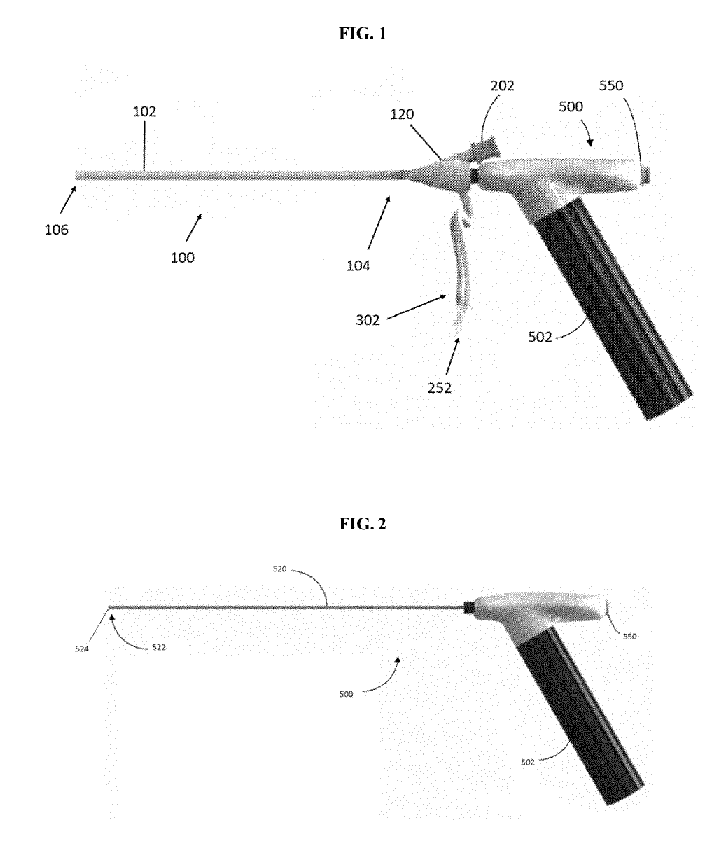

FIGS. 1-4 illustrate one embodiment of an imaging sleeve 100 with an imaging device 500. The imaging sleeve 100 includes a flexible elongate shaft 102 with an engagement member 120. The sleeve 100 can be engaged with the imaging device 500, similar to the devices illustrated in U.S. Patent App. Pub. No. 2017/0055813 to London Brown, et al., filed on Oct. 5, 2016, which is incorporated herein by reference in its entirety. The imaging device 500 can have a handle 502 and an elongate imaging member 520 extending distally from the handle 502. The handle 502 can include a light source disposed within the handle 502.

The elongate imaging member 520 extends from the handle 502 and is configured to be inserted through the engagement member 120 and into the elongate shaft 102. The elongate imaging member 520 has a distal portion 522 that terminates at a distal end of the elongate shaft 102 or at a point proximal to the distal end. The elongate imaging member 520 further includes an imaging unit 524 engaged on the distal portion 522 of the elongate imaging member 520. The imaging unit 524 can take a variety of forms, such as an active-pixel sensor array or a Complementary Metal-Oxide Semiconductor (CMOS) sensor, as provided in more detail in U.S. Patent App. Pub. No. 2017/0055813. The imaging unit 524 can be configured to be in communication with the handle 502 and can be configured to take images of areas adjacent thereto with assistance from a plurality of light transmission devices.

A plurality of light transmission devices extend from the light source in the handle 502, through a lumen defined by the elongate imaging member 520, and terminate at respective distal ends thereof disposed about the distal portion 522 of the elongate imaging member 520. The light transmission devices are typically arranged about the imaging unit 524. The light transmission devices are configured to receive the light from the light source and to transmit the light to the distal ends thereof to provide light to the area adjacent to the imaging unit 524 such that images can be taken within a body cavity of a patient. The light transmission devices can include fiber optic elements or light delivery fibers, and can be rigid or flexible to allow for bending or flexing within the elongate shaft 102.

The imaging device 500 can also be configured to connect to a power source. For example, a power source can be disposed in the handle 502, or the power source can be external to the device and connect to the handle 502. The power source can be arranged to be in electrical communication with the light source in the handle 502. A communication element 550 can also be operably engaged with the handle 502 such that the communication element 550 is in signal communication with the imaging unit 524 and configured to receive an image signal therefrom associated with an image captured by the imaging unit 524 or to communicate electrical power to the imaging unit 524. In addition, a display device for displaying the image, or a computer device for storing or analyzing the image, can be in communication with the communication element 550 via a wired communication arrangement or a wireless communication arrangement. All connections provided herein can be either wired or wireless connections.

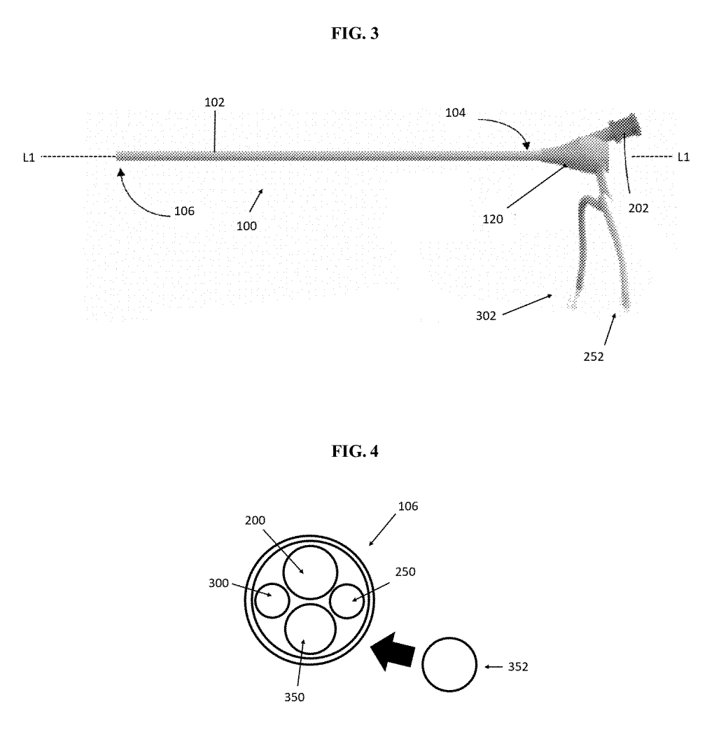

The elongate shaft 102 of the imaging sleeve 100 has the engagement member 120, a proximal end 104, a distal end 106, and one or more lumens that extend therethrough. As illustrated in FIGS. 3 and 4, four lumens 200, 250, 300, 350 extend therethrough, but any number of lumens can extend therethrough. Additionally, each lumen 200, 250, 300, 350 extends between the proximal end 104 and the distal end 106 of the elongate shaft 102. However, lumens can extend only partway between the proximal end 104 and the distal end 106. In some embodiments, the elongate shaft 102 can have an angled distal region that is angled at a non-zero angle relative to a longitudinal axis of the elongate shaft, such as being angled at approximately 15 degrees relative to the longitudinal axis of the elongate shaft.

The engagement member 120 is configured to engage the elongate shaft 102 on a distal end of the engagement member 120. The engagement member 120 is also configured to engage with the imaging device 500 on a proximal end of the engagement member 120. The engagement member 120 is configured to receive at least part of the imaging device 500 therethrough such that at least part of the imaging device 500 passes entirely therethrough and into a lumen of the elongate shaft 102. In some embodiments, the engagement member 120 is configured to secure the imaging device 500 with respect to the elongate shaft 102 such that the imaging device 500 is fixedly disposed in a distal position within the elongate shaft 102. As such, the imaging device 500 is non-slidable relative to and non-rotatable about a longitudinal axis L1 thereof within the elongate shaft 102. For example, the engagement member 102 can include one or more engagement fingers configured to engage corresponding features on the imaging device 500, such as in snap or friction fit engagements. The fingers can be configured to be engaged or disengaged by a user, such that the imaging sleeve 100 is removably, non-rotatably, and non-slidably affixed to the imaging device 500. Additionally, a variety of engagement features can be used, such as seals, clips, posts, locks, etc. While the sleeve 100 as illustrated is non-rotatable relative to the imaging device 500 when the engagement member is engaged, other embodiments can be provided with rotation means configured to rotate the sleeve 100 relative to the imaging device 500 without disengaging the sleeve 100 from the imaging device 500, for example by utilizing a rotation knob around the engagement member 120.

A working channel lumen 200 extends from the proximal end 104 to the distal end 106 and has openings on proximal and distal ends thereof. The working channel lumen 200 is configured to receive surgical instruments therethrough such that a surgical instrument can be inserted into the lumen 200 at the proximal end 104 so as to extend through and protrude from the distal end 106 of the elongate shaft 102. Surgical instruments can thus access, for example, a body cavity of a patient through the lumen 200. A distal end of the lumen 200 can be entirely open, allowing free passage of tools and materials therethrough. A proximal end of the lumen 200, however, can have various engagement mechanisms formed therein. For example, an engagement mechanism 202, such as a luer fitting, can be engaged with the proximal end of the lumen 200. The engagement mechanism 202 can be configured to engage, secure, and/or form a seal with the surgical instrument inserted into the working channel lumen 200.

Fluid inflow and fluid outflow lumens 250, 300 extend from the proximal end 104 to the distal end 106 of the elongate shaft 102 and have openings on proximal and distal ends thereof. The fluid inflow lumen 250 is configured to allow inflow of fluid to the body cavity, and the fluid outflow lumen 300 is configured to receive outflows of fluid from the body cavity. The fluid inflow lumen 250 engages an inflow irrigation tube 252, and the fluid outflow lumen 300 engages an outflow irrigation tube 302 to allow fluid to be provided and removed from the lumens 250, 300. One or more seals, engagements, mechanisms, valves, etc. can be disposed on proximal ends of the lumens 250, 300 to allow engagement with the tubes 252, 302, as explained further in U.S. Patent App. Pub. No. 2017/0055813 to London Brown, et al., filed on Oct. 5, 2016, which is incorporated herein by reference in its entirety.

A viewing lumen 350, which has an opening on the proximal end 104 of the elongate shaft 102, extends from the proximal end 104 to the distal end 106 of the elongate shaft 102. The viewing lumen 350 is configured to receive the imaging device 500 therein, and can be sealed at the distal end 106 of the elongate shaft, for example by a transparent member 352. The transparent member 352 is configured to allow imaging of the body cavity by the imaging device 500 therethrough and has an approximately circular shape. It extends across and seals only the viewing lumen, as illustrated in FIG. 3. However, it can have a variety of other configurations, such as having different shapes and sizes. Alternatively and/or additionally, a multi-lumen cap can be configured to be disposed on the distal end 106 of the elongate shaft, as discussed below.

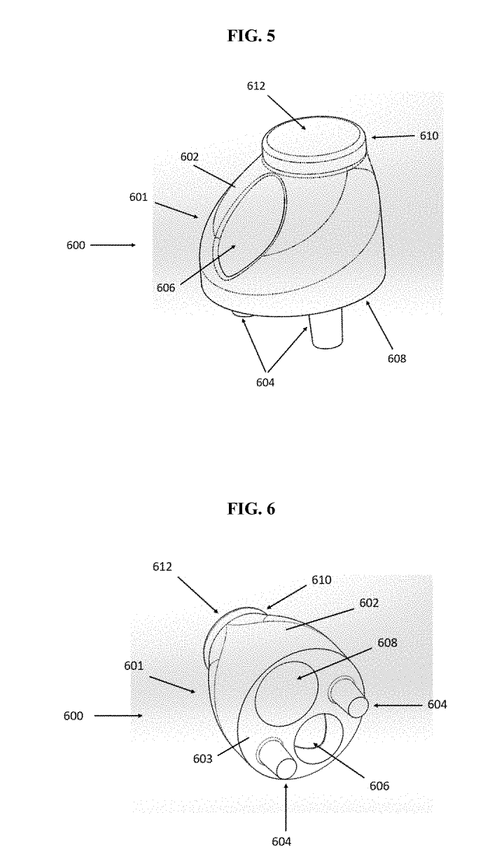

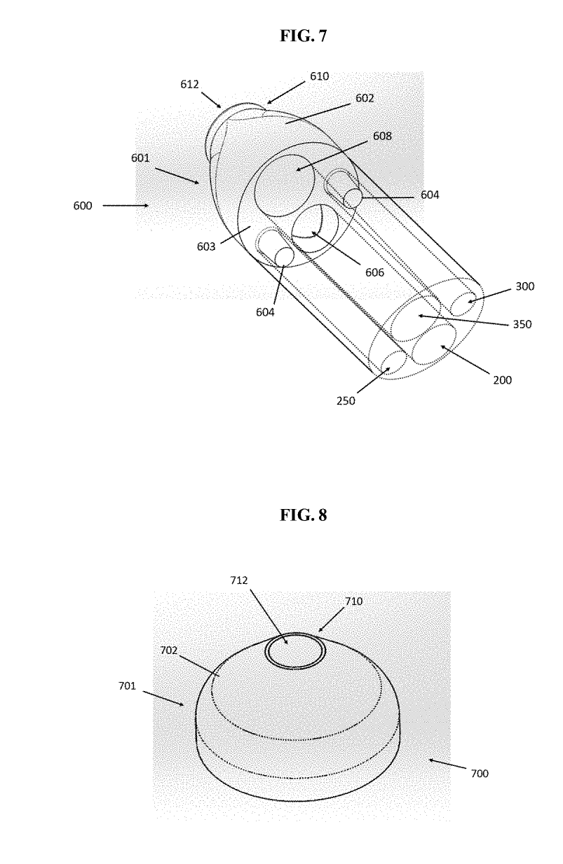

A multi-lumen cap can be configured to engage a distal end of an elongate shaft that contains more than one lumen to selectively seal one or more lumens while leaving other lumens open. For example, as illustrated in FIGS. 5-7, a cap 600 is configured to engage the distal end 106 of the elongate shaft 102 to selectively seal some lumens while leaving other lumens open. The cap 600 has a body 601 with an asymmetrical, conical shape, and the body 601 has a curved, asymmetrical, distal-facing surface 602 and a circular, proximal-facing flat surface 603 that is configured to sit across the distal end 106 of the elongate shaft 102 such that each of the lumens 200, 250, 300, 350 interacts with the cap 600. The cap 600 comes to a blunt point or tip at an engagement lid 610 that represents the distal-most feature of the cap 600, discussed in more detail below. The curved, blunt shape of the cap 600 is configured to reduce or eliminate shape edges thereon, while the blunt point or tip of the cap 600 formed by the lid 610 is configured to provide an easier insertion of the cap 600 and the elongate shaft 102 to the target tissue.

As illustrated, the cap 600 has two engagement protrusions 604 extending from the proximal-facing surface 603 that are sized and shaped to seal the fluid inflow and fluid outflow lumens 250, 300. The two protrusions 604 are cylindrical extensions that sit inside the lumens 250, 300 to seal the openings of the lumens 250, 300 at the distal end 106 of the elongate shaft 102.

An open channel 606 extends from the proximal-facing surface 603, through the body 601 of the cap 600, and to the distal-facing surface 602 with openings on each end, and the channel 606 is configured to align with the working channel lumen 200 such that it is sized and shaped to correspond to the working channel lumen 200. The open channel 606 is thus configured to allow any surgical instrument passed along the working channel lumen 200 to subsequently pass through the open channel 606 and into a body cavity of a patient.

A viewing channel 608 extends through the body 601 of the cap 600 from the proximal-facing surface 603 to the distal-facing surface 602, and it is configured to align with the viewing lumen 350 such that it is sized and shaped to correspond to the viewing lumen 350. The viewing channel 608 has an opening at the proximal-facing surface 603 but is sealed on the distal-facing surface 602 by the engagement lid 610 with a transparent window 612 therein. The transparent window 612 is similar to the transparent member 352. The transparent window 612 is an optically clear window that acts as both a protective sheath and a viral barrier for the imaging device 500 in the viewing lumen 350 or any other viewing apparatus that requires an unobstructed view of a target area. The lid 610 is fixed in place on the distal-most surface 602 of the cap 600. However, in other embodiments the lid can be configured to be removed and/or replaced. In an alternate embodiment, the viewing channel can be open on the distal end thereof without any lid.

The cap 600 engages the distal end 106 of the elongate shaft 102 through a variety of means, such as through friction fit between the protrusions 604 and the lumens 250, 300, through various binding means, through mechanical engagements, etc. Thus with the cap 600 in place on the distal end 106 of the elongate shaft 102, the fluid inflow and fluid outflow lumens 250, 300 are sealed by the engagement protrusions 604, the working channel lumen 200 is open to the body cavity therebeyond through the channel 606, and the viewing lumen 350 is sealed by the viewing channel 608 and the lid 610 while allowing imaging therethrough by the window 612. However, there are a variety of other possible configurations and uses of the multi-lumen caps disclosed herein. The multi-lumen caps can have any number of protrusions, open channels, and/or viewing channels therethrough and can be configured to engage with any number of devices other than just the sleeve 100 and the imaging device 500 described above. The multi-lumen caps provided herein can thus be designed to create any configuration of sealed and unsealed lumens of an apparatus that contains more than one channel or lumen. The multi-lumen caps can have one or more features that seal one or more channels or lumens of a corresponding device, as well as have features that maintain one or more unobstructed channels or lumens of the corresponding device. The features can be of any combination of length, size, and shape to correspond to the channels and/or lumens in the corresponding device. In addition to the ability to create any combination of sealed and unobstructed channels, the multi-lumen caps herein can have a blunt shape to eliminate any sharp edges and provide ease of insertion to any target area, and they can also be either symmetrical or asymmetrical depending on the desired use.

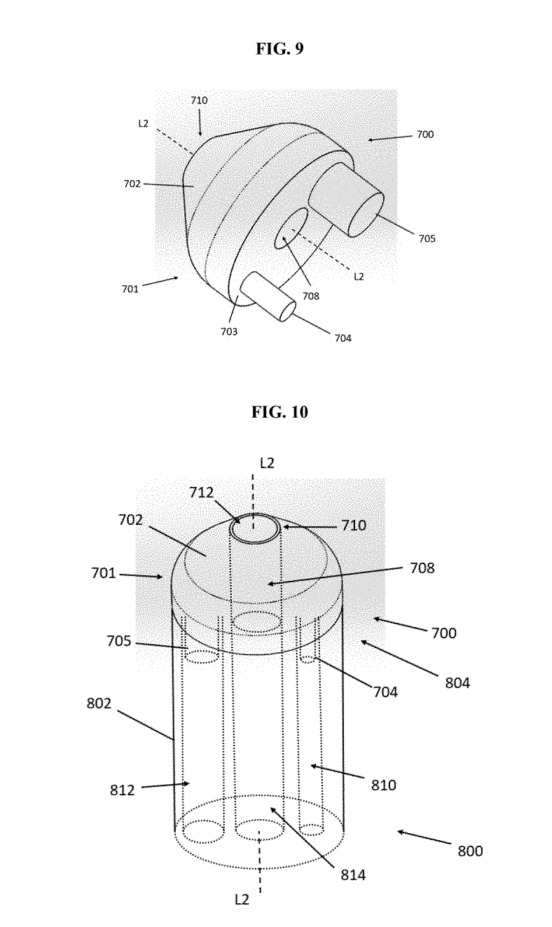

FIGS. 8-10 illustrate a cap 700 that is a symmetrical, blunted conical shape that has similar elements to the cap 600. The cap 700 is configured to engage a distal end 804 of an elongate shaft 802 of a corresponding device 800 to selectively seal some lumens. The cap 700 has a body 701 with a rounded, symmetrical, distal-facing surface 702 and a circular, proximal-facing flat surface 703 that is configured to sit across the distal end 804 of the elongate shaft 802. The cap 700 comes to a blunt point or tip at an engagement lid 710 that represents the distal-most feature of the cap 700. The rounded, blunt shape of the cap 700 is configured to reduce or eliminate sharp edges thereon, while the blunt distal-most point or tip of the cap 700 formed by the lid 710 is configured to provide an easier insertion of the cap 700 and the elongate shaft 802 to a desired target area.

The cap 700 has two engagement protrusions 704, 705 extending from the proximal-facing surface 703 that are sized and shaped to seal lumens 810, 812 that extend through the elongate shaft 802 of the corresponding device 800. The protrusions 704, 705 are two cylindrical extensions that sit inside the lumens 810, 812 to seal the openings of the lumens 810, 812 at the distal end 804 of the elongate shaft 802 and have different sizes than each other. However, protrusions can have any size, length, or shape as needed to seal lumens of a corresponding device.

A viewing channel 708 extends through the body 701 along a central longitudinal axis L2 of the cap 700 from the proximal-facing surface 703 to the distal-facing surface 702. The channel 708 is configured to align with a lumen 814 that extends through the elongate shaft 802 of the corresponding device 800, and it is sized and shaped to correspond to the lumen 814. The viewing channel 708 has an opening at the proximal-facing surface 703 but is sealed on the distal-facing surface 702 by the engagement lid 710 with a transparent window 712 therein. The transparent window 712 is similar to the transparent member 352 and is an optically clear window that can act as both a protective sheath and a viral barrier for any imaging device included with the corresponding device 800, for example the imaging device 500 that can be provided in the lumen 814. The lid 710 is fixed in place on the distal-most surface 702 of the cap 700. However, in other embodiments the lid could be configured to be removed and/or replaced. For example, the viewing channel can be open on the distal end thereof such that the lid and the window are entirely absent.

The cap 700 provided herein entirely seals the lumens 810, 812, 814 provided in the device 800, but in various embodiments, the caps can seal all lumens, can leave all lumens open, or any combination of the two. The cap 700 engages the distal end 804 of the elongate shaft 802 through a variety of means, such as through friction fit between the protrusions 704, 705 and the lumens 810, 812, through various binding means, through mechanical engagements, etc.

While the embodiments herein illustrate various cylindrical shafts for protrusions and hollow cylindrical shafts for lumens, the protrusions can be any combination of number, length, size, and shape, such as squares, ovals, stars, etc. and the corresponding lumens can be any combination of number, length, size, and shape, such as squares, ovals, stars, etc. The protrusions do not have to be an exact corresponding shape to the corresponding lumens as long as the protrusions can be received therein and can provide sufficient sealing force for the desired uses.

The multi-lumen caps discussed herein can be constructed using various manufacturing processes, such as injection molding, 3D printing, extruded then tipped, and/or mechanical construction, and they can be used on a variety of devices and incorporated into a variety of surgical tools, such as the sleeve 100 and the imaging device 500 discussed above. Additionally, the multi-lumen caps can be made from a variety of different materials, including Polyether ether ketone (PEEK), High-density polyethylene (HDPE), Polytetrafluoroethylene (PTFE), Polyether block amide (PEBA) such as PEBAX, other thermoplastic elastomers (TPE), silicone, epoxies, and/or Stainless Steel with any combination of polymers.

The multi-lumen caps sleeves disclosed herein can be provided in any of a variety of sizes, depending on patient anatomy, procedure type, imaging device to be used, and various other parameters that will be readily apparent to one having ordinary skill in the art. In some embodiments, the multi-lumen caps can engage with the sleeves disclosed herein that can have a variety of lengths, such as about 10 cm to 40 cm, and a variety of diameters, such as about 1 mm to 6 mm.

In the present disclosure, like-numbered components of the embodiments generally have similar features, and thus within a particular embodiment each feature of each like-numbered component is not necessarily fully elaborated upon. Sizes and shapes of the devices described herein, and the components thereof, can depend at least on the anatomy of the subject in which the devices will be used, the size and shape of components with which the devices will be used, and the methods and procedures in which the devices will be used. The figures provided herein are not necessarily to scale. Although the devices and methods disclosed herein are generally directed to surgical techniques, they can also be used in applications outside of the surgical field. Although the invention has been described by reference to specific embodiments, it should be understood that numerous changes may be made within the spirit and scope of the inventive concepts described. Accordingly, it is intended that the invention not be limited to the described embodiments, but that it have the full scope defined by the language of the following claims.

What is claimed is:

1. An imaging sleeve, comprising:
an elongate shaft having distal and proximal ends, a plurality of lumens extending therebetween, and a longitudinal axis extending therealong, the elongate shaft being configured to receive at least a portion of an imaging device therein;
an engagement member positioned on the proximal end of the elongate shaft and configured to engage with the imaging device; and
a cap configured to removably attach to the distal end of the elongate shaft and engage each of the plurality of lumens, wherein the cap is configured to seal one or more of the plurality of lumens and to leave open one or more of the plurality of lumens such that the cap either seals or leaves open each of the plurality of lumens.

2. The imaging sleeve of claim 1, wherein one or more protuberances extend proximally from a proximal-facing surface of the cap and are configured to correspond with and leave open one or more of the plurality of lumens.

3. The imaging sleeve of claim 1, wherein one or more openings extends through the cap and are configured to correspond with and seal one or more of the plurality of lumens.

4. The imaging sleeve of claim 1, further comprising a viewing lumen extending through the elongate shaft and a corresponding viewing channel extending through the cap, wherein the viewing lumen is configured to receive the imaging device therein.

5. The imaging sleeve of claim 4, wherein a distal end of the viewing channel of the cap is sealed and has a window disposed therein configured to allow imaging therethrough by the imaging device.

6. The imaging sleeve of claim 1, wherein the cap is configured to be asymmetrical.

7. The imaging sleeve of claim 1, wherein the cap is configured to be symmetrical.

8. The imaging sleeve of claim 1, further comprising fluid in and fluid out lumens extending through the elongate shaft and configured to deliver and remove fluid through the elongate shaft and to the distal end thereof, wherein the cap is configured to seal the fluid in and fluid out lumens.

9. The imaging sleeve of claim 1, further comprising a working lumen extending through the elongate shaft and configured to removably receive one or more surgical tools therethrough, wherein the cap is configured to leave open the working lumen.

10. An imaging system, comprising:
an imaging sleeve including an elongate shaft having distal and proximal ends and a plurality of lumens extending therebetween, the imaging sleeve having an engagement member positioned on the proximal end of the elongate shaft, and a cap being removably positioned on the distal end of the elongate shaft, the cap being configured to seal at least one of the plurality of lumens; and
an imaging device including a handle and an elongate imaging member extending distally from the handle, the elongate imaging member being configured to be inserted into one of the plurality of lumens of the elongate shaft of the imaging sleeve and the elongate imaging member being configured to take images therefrom, the handle being configured to engage with the engagement member of the imaging sleeve.

11. The imaging system of claim 10, wherein the lumen configured to receive the elongate imaging member through the elongate shaft is a viewing lumen, and a corresponding viewing channel extends through the cap, wherein the viewing channel is configured to allow imaging by the elongate imaging member therethrough.

12. The imaging system of claim 11, wherein a distal end of the viewing channel of the cap is sealed and has a window disposed therein configured to allow imaging therethrough by the elongate imaging member.

13. The imaging system of claim 10, wherein one or more protuberances extend proximally from a proximal-facing surface of the cap and are configured to correspond with and seal one or more of the plurality of lumens.

14. The imaging system of claim 10, wherein one or more openings extends through the cap and are configured to correspond with and leave open one or more of the plurality of lumens.

15. The imaging system of claim 10, wherein the engagement member is configured to prevent relative movement between the imaging sleeve and the imaging device.

16. The imaging system of claim 10, wherein the imaging device is a Complementary Metal-Oxide Semiconductor fiberscope.

17. A method of imaging a tissue sample, comprising:
attaching a cap onto a distal-most end of an imaging sleeve such that at least one of a plurality of lumens extending through the imaging sleeve is sealed by the cap;
inserting the cap and the imaging sleeve into a body cavity adjacent to tissue to be imaged;
imaging a tissue sample using an imaging device disposed at least partially in the imaging sleeve, the imaging device imaging the tissue through a window on the cap on the imaging sleeve.

18. The method of claim 17, further comprising sealing a viewing lumen that extends through the imaging sleeve by positioning a viewing channel and window in the cap to positionally correspond with the viewing lumen to allow the imaging device to image through the window, wherein the imaging device is at least partially disposed in the viewing lumen.

19. The method of claim 17, further comprising sealing fluid in and fluid out lumens that extend through the imaging sleeve by positioning protuberances on the cap into the lumens such that irrigation cannot be applied therethrough.

20. The method of claim 17, further comprising:
positioning an opening in the cap to correspond to a working lumen that extends through the imaging sleeve; and
passing a surgical tool through the working lumen and the opening to a surgical site.
